(19) United States
(12) Patent Application Publication
(10) Pub. No.: US 2025/0145580 A1
(43) Pub. Date: May 8, 2025

(54) AN IMPROVED PROCESS FOR THE PREPARATION OF FERRIC MALTOL

(71) Applicants: RK Pharma Inc., New York, NY (US); Apicore Pharmaceuticals Private Limited, Vadodara, Gujrat (IN)

(72) Inventors: Ravishanker KOVI, North Brunswick, NJ (US); Jayaraman KANNAPPAN, Vadodara, Gujrat (IN); Hemant MANDE, Vadodara, Gujrat (IN); Srikrishna APAR, Vadodara, Gujrat (IN)

(21) Appl. No.: 18/693,964

(22) PCT Filed: May 26, 2022
(86) PCT No.: PCT/IB2022/054961

§ 371 Date: Mar. 21, 2024

(30) Foreign Application Priority Data
Sep. 21, 2021 (IN) 202141042716
Feb. 18, 2022 (IN) 202241008728

(51) Int. Cl. C07D 309/40

(52) U.S. Cl. CPC C07D 309/40

(57) ABSTRACT

The present invention provides a new and improved method of forming Ferric maltol by reacting a Maltol in an organic alkaline solution/slurry with a non-carboxylate iron salt in an organic solvent. The present invention also provides an improved process for the preparation of polymorphic form alpha of ferric maltol. The present invention further provides novel polymorphic forms RK1, RK2, RK3, RK4, RK5, RK6 and RK7 of Ferric maltol with a characteristic XRPD Patterns respectively and process for their preparation thereof.

### RELATED APPLICATION

This application is a PCT International application and claims priority to and the benefit of Indian Provisional Patent Application Number 202141042716 dated 21 Sep. 2021 and claims the priority to and the benefit of Indian Provisional Patent Application Number 202241008728 dated 18 Feb. 2022, the contents of which are incorporated herein in its entirety.

### FIELD OF THE INVENTION

The present invention relates to an improved process for the preparation of ferric maltol or ferric trimaltol. The present invention also relates to the novel polymorphic forms RK1, RK2, RK3, RK4, RK5, RK6 and RK7 of Ferric maltol and process for their preparation.

### BACKGROUND OF THE INVENTION

ACCRUFER (ferric maltol) is used as an iron replacement product for oral administration. Accrufer is available in capsule form which contains 30 mg iron and 201.5 mg of maltol. Ferric maltol contains iron in a stable ferric state as a complex with a trimaltol ligand. Ferric maltol is 3-hydroxy-2-methyl-4H-pyrane-4-one iron (III) complex (3:1) and has the molecular formula (C₆H₅O₃)₃Fe and a molecular mass of 431.2 g/mol.

Each red capsule, printed with “30”, contains colloidal anhydrous silica, crospovidone (Type A), lactose monohydrate, magnesium stearate and sodium lauryl sulfate as inactive ingredients. In addition, the capsule shell contains FD&C Blue No. 1, FD&C Red No. 40, FD&C Yellow No. 6, gelatin and titanium dioxide. The ink used for printing the marking contains ammonium hydroxide, ethanol, iron oxide black and propylene 25 glycol.

U.S. Pat. No. 4,575,502 Patent provides the preparation of ferric maltol by mixing a chloroform solution of maltol with a 1M solution of ferric chloride in ethanol to provide a 3:1 molar ratio of maltol:iron in the mixture, followed by addition of excess of solid sodium carbonate and stirring the mixture for 10 minutes, filtering the mixture and evaporating the solvent to give the neutral complex containing maltol and the ferric cation in 3:1 proportion. Recrystallization was carried out from ethanol to give wine red needle crystals. However, this method suffers from the drawback that use of chloroform is not preferable from the scale up point of view. Our present invention uses dichloromethane which is commercially suitable.

U.S. Pat. No. 5,051,523 Patent provides the preparation of ferric maltol by adding iron (III) acetylacetonate in ethyl acetate to suspension of maltol in water, stirring for 3 hours at rt, extracting the aqueous phase repeatedly with ethyl acetate and thereafter lyophilizing to give the iron (III) maltol complex. However, ferric maltol is soluble in aqueous phase and multiple extractions are required to isolate it. Our present invention doesn't require extraction for isolating ferric maltol. In addition, aqueous phase reaction forms ferric hydroxide which is insoluble in nature and acts as impurity.

U.S. Pat. No. 7,459,569 Patent provides the preparation of ferric maltol by adding non-ferric salt of a C1 to C12 carboxylic acid such as ferric citrate to NaOH solution and then adding ferric citrate solution to maltol to form ferric trimaltol. However, several of the iron carboxylate salts employed are expensive, especially of pharmaceutical grade. In addition, this process introduces high levels of carboxylates to the synthesis that are not easily removed by filtration or centrifugation of the ferric trimaltol cake. Instead, these water-soluble contaminants must be washed off (e.g., water washed), but this would result in considerable losses of the product due to the amphipathic nature of ferric trimaltol. In addition, aqueous phase reaction forms ferric hydroxide which is insoluble in nature and acts as impurity. However, our present invention doesn't use iron carboxylate salts and process is non-aqueous, commercially scalable and economical.

U.S. Pat. No. 9,096,629 Patent provides the preparation of ferric maltol by combining maltol with an aqueous solution having a pH of 3 to about 7; heating the combined maltol and aqueous solution to form a heated maltol solution having a pH of 3 to 7; combining the heated maltol solution with a non-carboxylate iron salt to form a solution having a pH of 3 to 7; combining the solution having a pH of 3 to 7 with an aqueous alkaline solution comprising maltol, wherein the aqueous alkaline solution has a pH of greater than 7; and precipitating iron tri(maltol) from the combined solutions having a pH of greater than 7. As mentioned earlier, aqueous phase reaction forms Ferric hydroxide which is insoluble in nature and acts as impurity. Also, yield obtained is low, whereas our present invention provides a process which is non-aqueous, commercially scalable and economical.

Chemistry for Sustainable Development 15 (2007) 448-458 discloses preparation of ferric maltol by addition of solution of FeCl₃·6H₂O in water to the solution of maltol in water followed by addition of 2M NaOH solution; followed by stirring, evaporating in a rotary evaporator and extracting the dry residue with chloroform to remove the inorganic part, which is insoluble in chloroform and then evaporating the chloroform extract to the dry state and purifying the resulting complex by re-precipitation from methanol to diethyl ether and separating the precipitated small purple crystals by filtering and drying under reduced pressure. This method suffers from the drawback of formation of ferric hydroxide and ferrous salt as impurity.

Present invention provides a new and improved method of forming an iron hydroxypyrone Ferric Maltol compound. In comparison to prior arts described above, our present invention provides particular advantages such as
1. Use of Non aqueous system: Due to non-aqueous system, formation of Fe(OH)₃ is completely avoided.
2. Ferrous content: Ferrous content formation is controlled or not detected as present invention involves non-aqueous solvent system.
3. High yield: Yield of 80-90% can be obtained by this method.
4. Cost effective and commercially viable
5. High Purity: Product obtained is having high purity (around 99.9%)

Polymorphism, the occurrence of different crystal form is a property of some molecules and molecular complexes. Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Discovering new salts, solid state forms and solvates of a pharmaceutical product can provide materials having desirable processing properties, such as ease of handling, ease of processing, storage stability, and ease of purification or as desirable intermediate crystal forms that facilitate conversion to other salts or polymorphic forms.

U.S. Pat. No. 9,802,973, which is incorporated herein by reference, has claimed four different polymorphic forms of ferric maltol namely form I, form II, form III and form IV. It further discusses the process for the preparation of these forms. Same patent further covers use of these polymorphic forms for the preparation of pharmaceutical composition.

US 2021/0139518A1 Patent application provides a novel crystalline ferric maltol alfa form characterized by a XRPD pattern having peaks at 9.4, 12.7, 14.4, 15.2, 17.3, 19.8, 21.1, 23, 24.3° 2θ±0.2° 2θ.

Since improved drug formulations are consistently sought, there is an ongoing need for new and pure polymorphic forms of existing drug molecules. For at least these reasons, there is a need for additional solid state forms (including solvated forms) of ferric maltol.

Present invention provides novel polymorphic forms RK1, RK2, RK3, RK4, RK5, RK6 and RK7 of Ferric maltol and also provide process for their preparation.

### SUMMARY OF THE INVENTION

In a first aspect, the present invention provides a new and improved method of forming Ferric maltol comprising the steps of
a) forming a slurry of maltol by dissolving/slurring alkali in organic solvent and then introducing maltol and stirring;
b) reacting an organic alkaline slurry of maltol with a non-carboxylate iron salt to obtain solution or slurry of Ferric maltol;
c) adding anti-solvent optionally in the case when Ferric maltol obtained in step b) is in solution form;
d) isolating Ferric maltol from the reaction vessel.

In a second aspect, the present invention provides an improved process for the preparation of polymorphic form alpha of ferric maltol characterized by a PXRD pattern as illustrated in FIG. 22 having characteristic crystalline peaks at 9.4, 12.7, 14.4, 15.2, 17.3, 19.8, 21.1, 23, 24.3° 2θ±0.2° 2θ, comprising the steps of:
a) providing a solution of ferric maltol in an organic solvent;
b) addition of anti-solvent to the solution obtained in step a);
c) isolation of polymorphic Form Alfa of Ferric maltol

In a third aspect, the present invention provides another improved process for the preparation of polymorphic form alpha of ferric maltol characterized by a PXRD pattern as illustrated in FIG. 22 having characteristic crystalline peaks at 9.4, 12.7, 14.4, 15.2, 17.3, 19.8, 21.1, 23, 24.3° 2θ±0.2° 2θ, comprising the steps of:
a) forming a solution or slurry of maltol by dissolving/slurring alkali in organic solvent and then introducing maltol and stirring;
b) reacting an organic alkaline solution or slurry of maltol with a non-carboxylate iron salt to obtain solution of Ferric maltol;
c) addition of anti-solvent to the solution obtained in step b);
d) isolation of polymorphic Form Alfa of Ferric maltol from the reaction vessel.

In a fourth aspect, the present invention provides novel polymorphic form RK1 ferric maltol that can be characterized by X-ray powder diffraction (XRPD) pattern, differential scanning calorimetry (DSC) and Thermogravimetric analysis (TGA) as illustrated in FIG. 1, FIG. 2 and FIG. 3 respectively and also provides process for its preparation.

In a fifth aspect, the present invention also provides novel polymorphic form RK2 ferric maltol that can be characterized by its X-ray powder diffraction (XRPD) pattern, differential scanning calorimetry (DSC) and Thermogravimetric analysis (TGA) as illustrated in FIG. 4, FIG. 5 and FIG. 6 respectively and also provides process for its preparation.

In a sixth aspect, the present invention provides novel polymorphic form RK3 of ferric maltol which is a Dichloromethane solvate that can be characterized by X-ray powder diffraction (XRPD) pattern, differential scanning calorimetry (DSC) and Thermogravimetric analysis (TGA) as illustrated in FIG. 7, FIG. 8 and FIG. 9 respectively and also provides process for its preparation.

In a seventh aspect, the present invention provides novel polymorphic form RK4 of ferric maltol which is a Chlorobenzene solvate that can be characterized by X-ray powder diffraction (XRPD) pattern, differential scanning calorimetry (DSC) and Thermogravimetric analysis (TGA) as illustrated in FIG. 10, FIG. 11 and FIG. 12 respectively and also provides process for its preparation.

In an eighth aspect, the present invention provides novel polymorphic form RK5 of ferric maltol which is a Carbon tetrachloride (CCl₄) solvate that can be characterized by X-ray powder diffraction (XRPD) pattern, differential scanning calorimetry (DSC) and Thermogravimetric analysis (TGA) as illustrated in FIG. 13, FIG. 14 and FIG. 15 respectively and also provides process for its preparation.

In a ninth aspect, the present invention provides novel polymorphic form RK6 of ferric maltol which is an Acetic Acid Hemi solvate that can be characterized by X-ray powder diffraction (XRPD) pattern, differential scanning calorimetry (DSC) and Thermogravimetric analysis (TGA) as illustrated in FIG. 16, FIG. 17 and FIG. 18 respectively and also provides process for its preparation.

In a tenth aspect, the present invention provides novel polymorphic form RK7 of ferric maltol which is a Propionic acid hemi solvate that can be characterized by X-ray powder diffraction (XRPD) pattern, differential scanning calorimetry (DSC) and Thermogravimetric analysis (TGA) as illustrated in FIG. 19, FIG. 20 and FIG. 21 respectively and also provides process for its preparation.

### BRIEF DESCRIPTION OF THE DRAWINGS

FIG. 1: XRPD of polymorphic form RK1 of ferric maltol

FIG. 2: DSC of polymorphic form RK1 of ferric maltol

FIG. 3: TGA of polymorphic form RK1 of ferric maltol

FIG. 4: XRPD of polymorphic form RK2 of ferric maltol

FIG. 5: DSC of polymorphic form RK2 of ferric maltol

FIG. 6: TGA of polymorphic form RK2 of ferric maltol

FIG. 7: XRPD of polymorphic form RK3 of ferric maltol

FIG. 8: DSC of polymorphic form RK3 of ferric maltol

FIG. 9: TGA of polymorphic form RK3 of ferric maltol

FIG. 10: XRPD of polymorphic form RK4 of ferric maltol

FIG. 11: DSC of polymorphic form RK4 of ferric maltol

FIG. 12: TGA of polymorphic form RK4 of ferric maltol

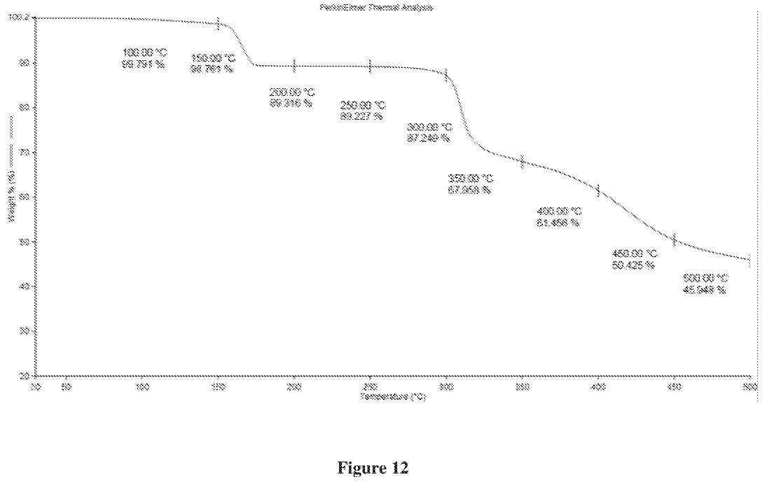

FIG. 13: XRPD of polymorphic form RK5 of ferric maltol

FIG. 14: DSC of polymorphic form RK5 of ferric maltol

FIG. 15: TGA of polymorphic form RK5 of ferric maltol

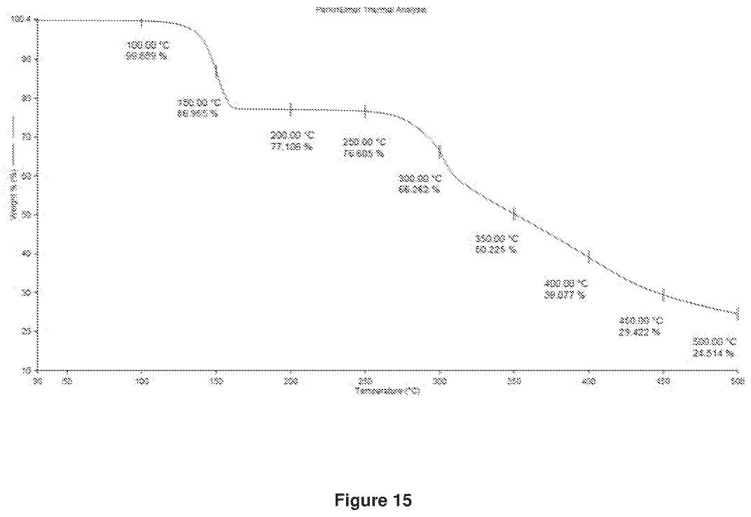

FIG. 16: XRPD of polymorphic form RK6 of ferric maltol

FIG. 17: DSC of polymorphic form RK6 of ferric maltol

FIG. 18: TGA of polymorphic form RK6 of ferric maltol

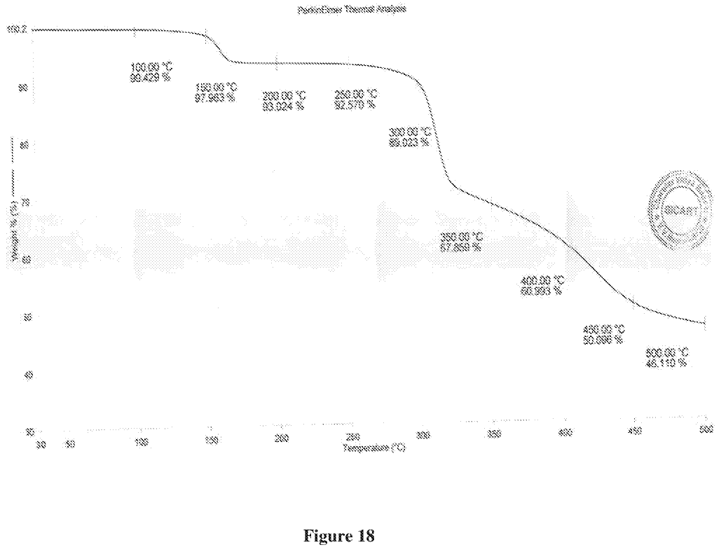

FIG. 19: XRPD of polymorphic form RK7 of ferric maltol

FIG. 20: DSC of polymorphic form RK7 of ferric maltol

FIG. 21: TGA of polymorphic form RK7 of ferric maltol

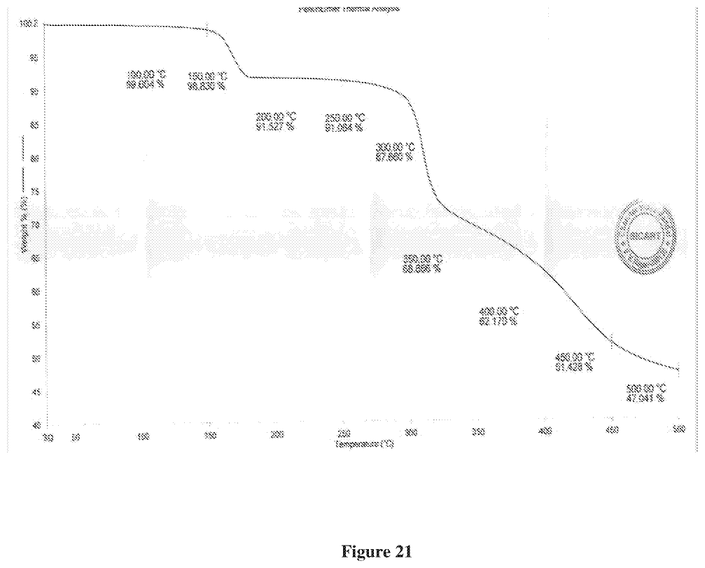

FIG. 22: X-ray crystal structure of Ferric Maltol Form Alpha

FIG. 23: PXRD comparison of simulated vs experimental patterns of Ferric Maltol Form Alpha.

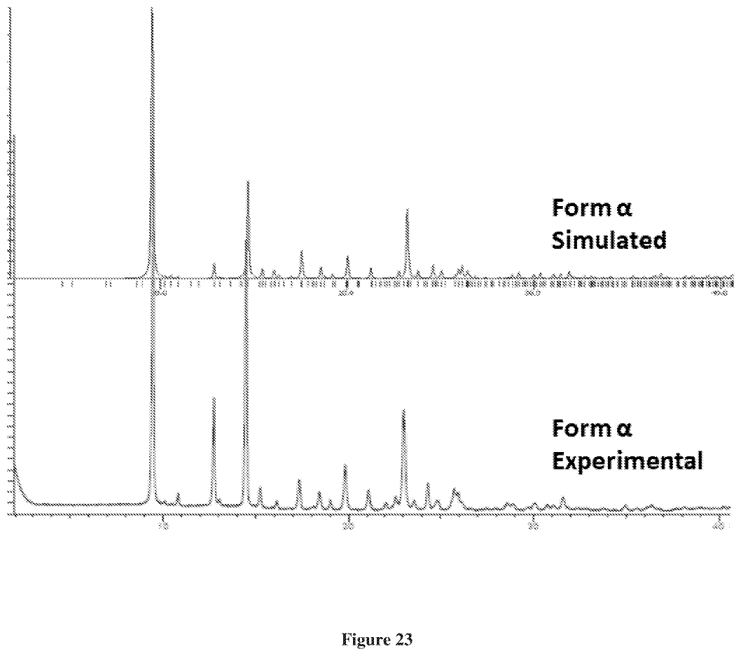

### DETAILED DESCRIPTION OF THE INVENTION

In a first embodiment, the invention provides a new and improved process for the preparation of ferric maltol comprising the steps of:
a) forming an organic alkaline solution or slurry of maltol by dissolving or slurring the alkali in an organic solvent, introducing maltol and then stirring;
b) reacting an organic alkaline solution or slurry of maltol with a non-carboxylate iron salt to obtain solution or slurry of Ferric maltol;
c) adding anti-solvent optionally in the case when Ferric maltol obtained in step b) is in solution form;
d) isolating Ferric maltol from the reaction vessel.

In a preferred embodiment of the invention, organic alkaline solution or slurry of maltol is formed at 10-40° C., preferably at room temperature.

In a further preferred embodiment of the invention, organic alkaline solution or slurry of maltolis cooled to 0-20° C., preferably to 10-20° C. and reacted at the same temperature with non-carboxylate iron salt by stirring for 2-4 hours, preferably for 2-3 hours.

In another embodiment of the invention, organic solvent used for forming organic alkaline solution or slurry of maltol can be selected from halogenated solvent such as dichloromethane, chloroform, carbon tetrachloride, chlorobenzene, 1,2-dichlorobenzene; polar aprotic solvent such as dimethylformamide, dimethylacetamide, dimethyl sulfoxide; alcoholic solvent such as methanol, ethanol, isopropanol or butanol.

In a preferred embodiment of the invention, organic solvent is selected from methanol, ethanol or dichloromethane.

In a further embodiment of the invention, alkali used for forming an organic alkaline solution or slurry of maltol can be selected from hydroxides, including alkali metal hydroxides, such as sodium hydroxide, potassium hydroxide or ammonium hydroxide; preferably sodium hydroxide.

In one embodiment, the base does not comprise a carbonate, such as sodium carbonate, or a hydrogen carbonate.

In another preferred embodiment of the invention, non-carboxylate iron salt may be selected from ferric chloride hexahydrate or ferric chloride anhydrous.

In a further preferred embodiment of the invention, anti-solvent added in step c) may be selected from ether solvent such as tetrahydrofuran, dioxane, diisopropyl ether, methyl tertiary butyl ether or diethyl ether, preferably methyl tertiary butyl ether (MTBE).

In a further embodiment of the invention, formed ferric maltol compound is separated by using any suitable technique known in the art such as, for example, filtration, for example, filtration under ambient or reduced pressure (for example, less than 1 bar) or under vacuum, or by centrifugation or decanting.

In a second embodiment of the invention, present invention provides Single Crystal Data of Polymorphic Form Alpha of Ferric Maltol

TABLE 1
Crystal data and structure refinement
for Ferric Maltol Form Alpha.

| | |
|---|---|
| Empirical formula | C₁₈H₁₅FeO₉ |
| Formula weight | 431.2 |
| Temperature/K | 297 |
| Crystal system | Orthorhombic |
| Space group | Pbca |
| a/Å | 16.875(3) |
| b/Å | 12.132(2) |
| c/Å | 18.335(2) |
| α/° | 90 |
| β/° | 90 |
| γ/° | 90 |
| Volume/Å³ | 3699.3(10) |
| Z | 8 |
| ρ_{calc} g/cm³ | 1.3631 |
| μ/mm⁻¹ | 0.850 |
| F(000) | 1554.6 |
| Crystal size/mm³ | 0.19 × 0.08 × 0.03 |
| Radiation | Mo Kα (λ = 0.71073) |
| Reflections collected | 2126 |
| Independent reflections | 263 [Rᵢₙₜ = 0.0307, R_{sigma} = 0.0173] |
| Data/restraints/parameters | 263/0/267 |
| Final R indexes [I >= 2σ (I)] | R₁ = 0.8960, wR₂ = 0.9537 |
| Final R indexes [all data] | R₁ = 0.8960, wR₂ = 0.9537 |

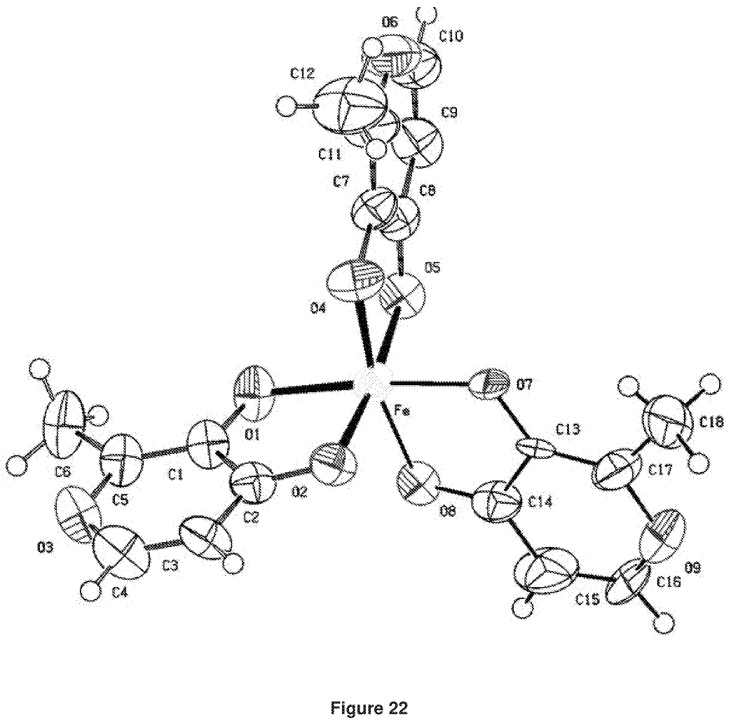

In a preferred embodiment, invention provides improved process for the preparation of polymorphic form alpha of ferric maltol characterized by a PXRD pattern as illustrated in FIG. 22 having characteristic crystalline peaks at 9.4, 12.7, 14.4, 15.2, 17.3, 19.8, 21.1, 23, 24.3° 2θ±0.2° 2θ, comprising the steps of:
a) providing a solution of ferric maltol in an organic solvent;
b) addition of anti-solvent to the solution obtained in step a);
c) isolation of polymorphic form Alfa of Ferric maltol

In a further preferred embodiment of the invention, polymorphic form alpha of ferric maltol characterized by a PXRD pattern as illustrated in FIG. 22 having characteristic crystalline peaks at 9.4, 12.7, 14.4, 15.2, 17.3, 19.8, 21.1, 23, 24.3° 2θ±0.2° 2θ, can also be synthesized by process comprising the steps of:
a) forming a solution or slurry of maltol by dissolving/slurring alkali in organic solvent and then introducing maltol and stirring;
b) reacting an organic alkaline solution or slurry of maltol with a non-carboxylate iron salt to obtain solution of Ferric maltol;
c) addition of anti-solvent to the solution obtained in step b);
d) isolation of polymorphic form alfa of Ferric maltol from the reaction vessel.

In a preferred embodiment of the invention, organic solvent used can be selected from halogenated solvent such as dichloromethane, chloroform, carbon tetrachloride, 1,2-dichlorobenzene, preferably, dichloromethane.

In another preferred embodiment of the invention, anti-solvent may be selected from ether solvent such as tetrahydrofuran, dioxane, Di isopropyl ether, methyl tertiary butyl ether or diethyl ether, preferably, methyl tertiary butyl ether (MTBE)

In a further preferred embodiment of the invention, before addition of anti-solvent, solution of ferric maltol in an organic solvent is filtered through 0.45-micron filter paper to remove traces of sodium chloride.

In a further embodiment of the invention, obtained ferric maltol is converted into polymorphic forms RK1, RK2, RK3, RK4, RK5, RK6 and RK7 of ferric maltol.

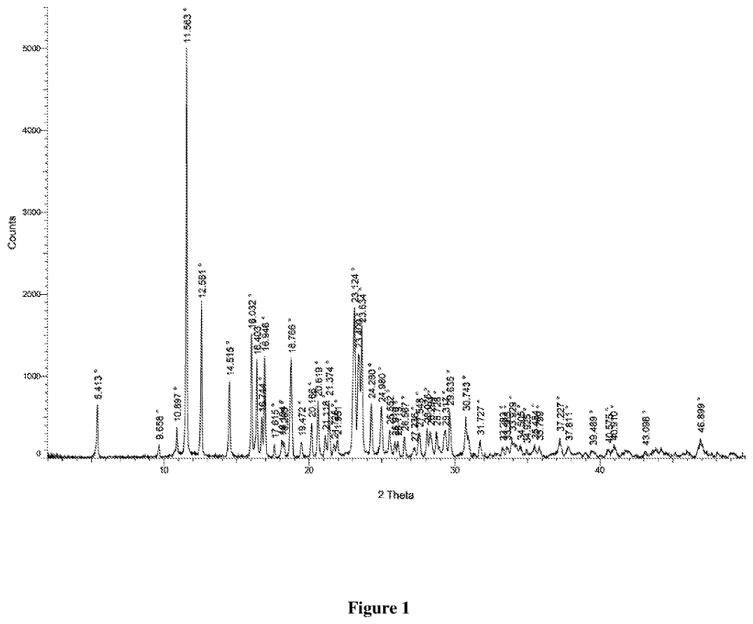

In a third embodiment, present invention provides a novel polymorphic form RK1 of ferric maltol which is a chloroform solvate characterized by XRPD as illustrated in FIG. 1.

In a preferred embodiment of the invention, polymorphic form RK1 can be characterized by XRPD comprising characteristic peaks at 5.4, 11.5, 12.5, 16.0, 16.4, 23.4 and 24.2±0.2° 2θ

In a further embodiment of the invention, XRPD of polymorphic form RK1 further shows additional peaks at 14.5, 16.9, 18.7, 20.6, 21.3, 23.1, 23.6 and 24.9±0.2° 2θ.

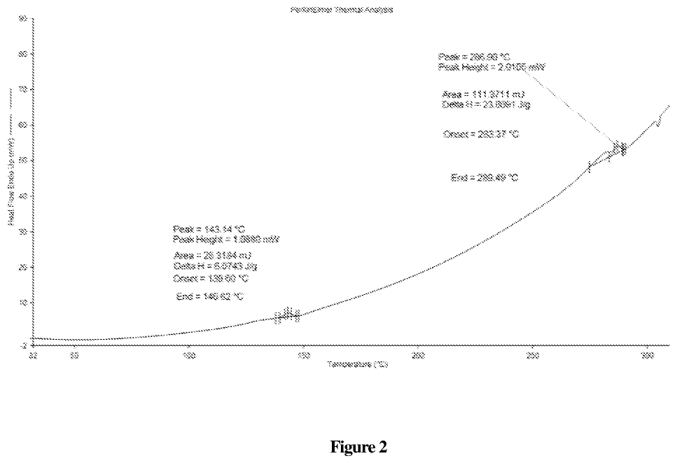

In another embodiment of the invention, polymorphic form RK1 can be characterized by DSC wherein peaks are obtained at 143.14° C. and 286.90° C. An illustrative DSC thermogram of polymorphic form RK1 is displayed in FIG. 2.

In a preferred embodiment of the invention, polymorphic form RK1 of ferric maltol can be obtained by
a) Solubilizing ferric maltol in chloroform or mixture of chloroform and organic solvent;
b) addition of anti-solvent to the solution obtained in step a);
c) isolation of polymorphic form RK1 of Ferric maltol from the reaction vessel.

In another preferred embodiment of the invention, ferric maltol was solubilized in chloroform or mixture of chloroform and organic solvent by stirring at room temperature to dissolve it completely.

In a further embodiment of the invention, solution obtained in step a) is filtered to remove undissolved particulate before addition of anti-solvent is carried out in step b)

In another preferred embodiment of the invention, organic solvent used in step a) may be selected from alcohol such as methanol, ethanol, 1-propanol, 2-propanol, 1-butanol, isobutanol and benzyl alcohol, preferably ethanol.

In a further preferred embodiment of the invention, anti-solvent used in step b) may be selected from ester such as ethyl acetate, butyl acetate, propyl acetate, isopropyl acetate; aliphatic ether such as methyl tertiary butyl ether, dimethyl ether, diethyl ether, diisopropyl ether; cyclic ether such as tetrahydrofuran and alkane such as hexane and heptane.

In a most preferred embodiment of the invention, polymorphic form RK1 of ferric maltol is obtained by solubilizing ferric maltol in mixture of chloroform and ethanol, filtering the solution to remove undissolved particulate, followed by addition of methyl tertiary butyl ether as anti-solvent.

In another preferred embodiment of the invention, polymorphic form RK1 of ferric maltol may be obtained by solubilizing ferric maltol in chloroform, filtering the solution to remove undissolved particulate and further adding ethyl acetate as anti-solvent.

In a further embodiment of the invention, polymorphic form RK1 of ferric maltol is converted into polymorphic form RK2 of ferric maltol.

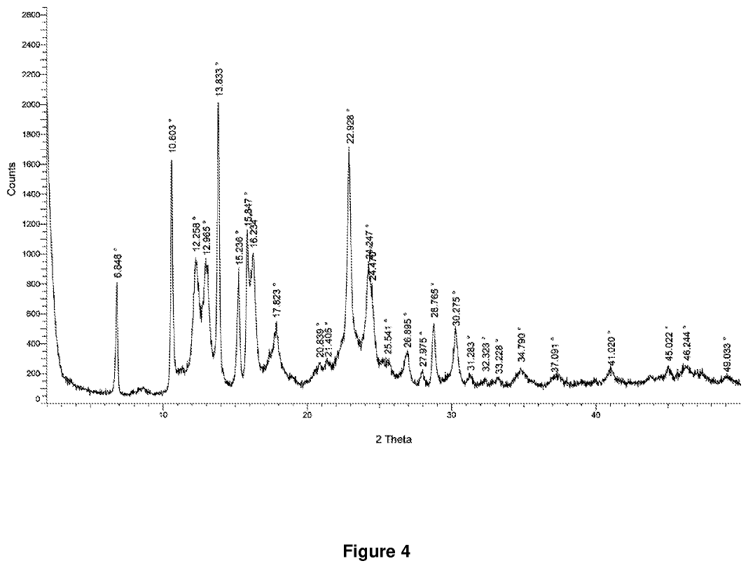

In a fourth embodiment, present invention provides a novel polymorphic form RK2 of Ferric maltol characterized by XRPD illustrated in FIG. 4.

In a preferred embodiment of the invention, polymorphic form RK2 can be characterized by XRPD comprising characteristic peaks at 6.8, 10.6, 12.2, 13.83, 15.23, 15.84, 22.9, 24.2±0.2° 2θ.

In a further embodiment of the invention, XRPD of polymorphic form RK2 further shows additional peaks at 12.9, 16.2, 17.8, 28.7, 30.2±0.2° 2θ.

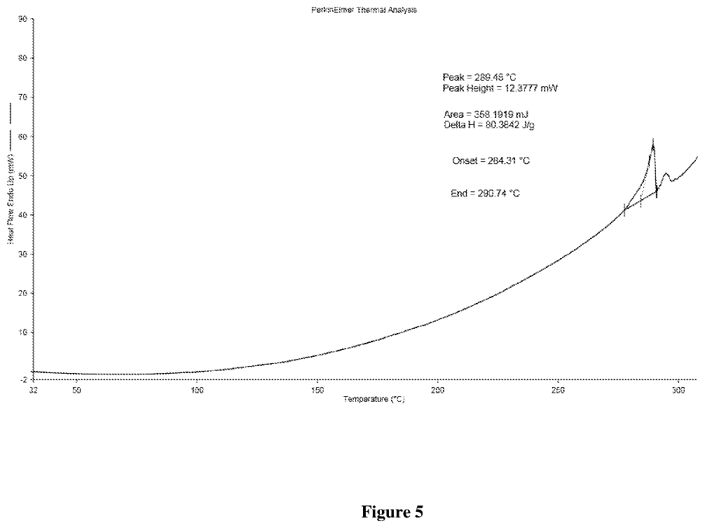

In another embodiment of the invention, polymorphic form RK2 can be characterized by DSC wherein single peak is obtained at 289.4° C. An illustrative DSC thermogram of polymorphic form RK2 is displayed in FIG. 5.

In a preferred embodiment, present invention provides process for the preparation of polymorphic form RK2 of ferric maltol comprising the step of drying the polymorphic form RK1 of ferric maltol at a temperature between 100° C.-150° C. for 3-4 hours.

In a further embodiment of the invention, polymorphic form RK2 of ferric maltol is further used for making pharmaceutical composition comprising ferric maltol.

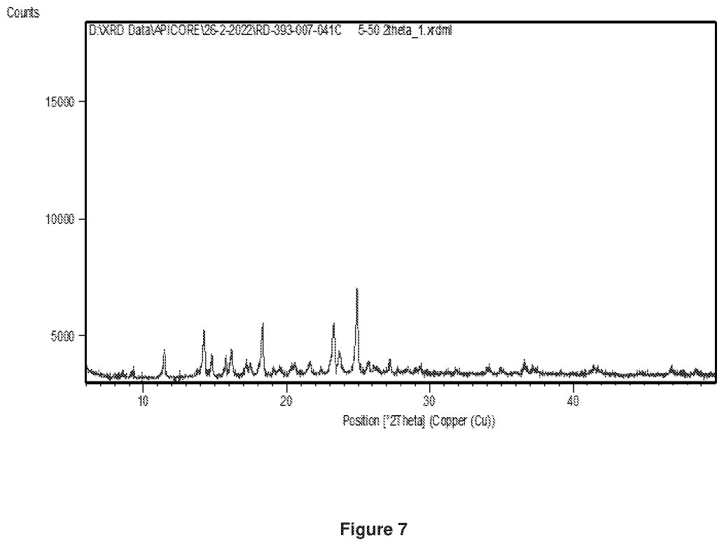

In a fifth embodiment, present invention provides a novel polymorphic form RK3 (Dichloromethane solvate) of Ferric maltol characterized by XRPD illustrated in FIG. 7.

In a preferred embodiment of the invention, polymorphic form RK3 which is a Dichloromethane solvate of Ferric maltol can be characterized by XRPD comprising characteristic peaks at 11.5, 14.2, 18.3, 23.3, 24.9±0.2° 2θ.

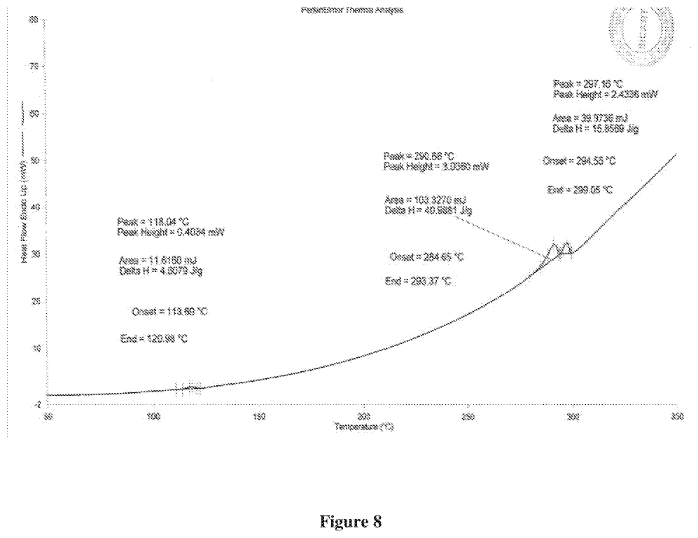

In another embodiment of the invention, polymorphic form RK3 can be characterized by DSC wherein three peaks are obtained at 118° C., 290° C. and 297° C. An illustrative DSC thermogram of polymorphic form RK3 is displayed in FIG. 8.

In a further embodiment, present invention provides process for the preparation of polymorphic form RK3 of Ferric maltol comprising the steps of.
a) Slurring the Ferric maltol in Dichloromethane at room temperature;
b) Adding Methyl tertiary butyl ether (MTBE) into the slurry mass at room temperature to obtain polymorphic form RK3 of Ferric maltol

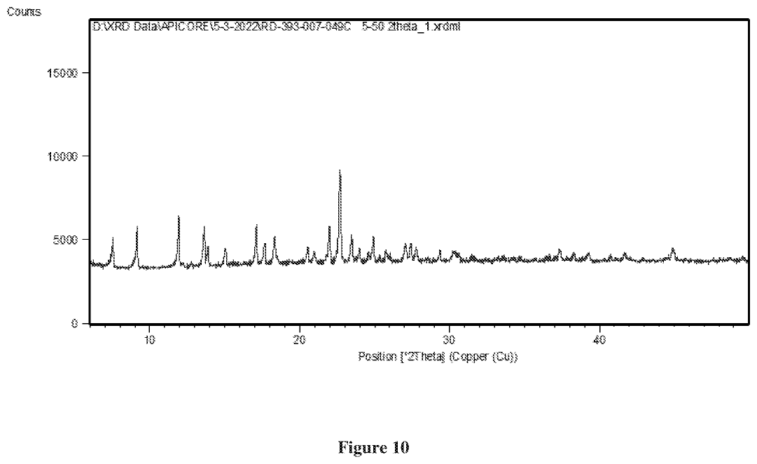

In a sixth embodiment, present invention provides a novel polymorphic form RK4 (Chlorobenzene Solvate) of Ferric maltol characterized by XRPD illustrated in FIG. 10.

In a preferred embodiment of the invention, polymorphic form RK4 which is a Chlorobenzene solvate of Ferric maltol can be characterized by XRPD comprising characteristic peaks at 7.5, 9.1, 11.9, 13.6, 17.1, 22.0 and 22.7±0.2° 2θ.

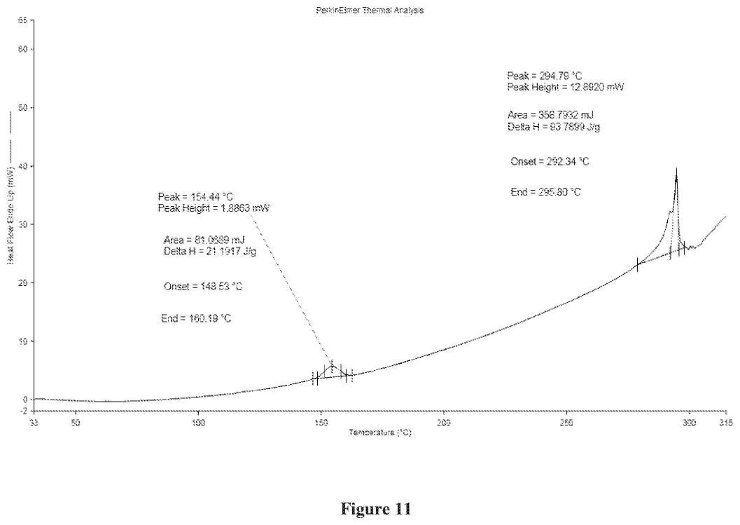

In another embodiment of the invention, polymorphic form RK4 can be characterized by DSC wherein two peaks are obtained at 154.4° C., and 294.7° C. An illustrative DSC thermogram of polymorphic form RK4 is displayed in FIG. 11.

In a further embodiment, present invention provides process for the preparation of polymorphic form RK4 of Ferric maltol comprising the steps of.
a) Dissolving Ferric maltol in Dichloromethaneor Chloroform at room temperature;
b) Filtering the solution to remove undissolved particulate;
c) evaporating the solvent from the filtrate on rotary evaporator to obtain Polymorphic form Alpha of ferric maltol;
d) Adding Chlorobenzene and stirring at room temperature for 5-10 minutes;
e) Adding Methyl tertiary butyl ether (MTBE) into the slurry mass at room temperature to obtain polymorphic form RK4 of Ferric malto

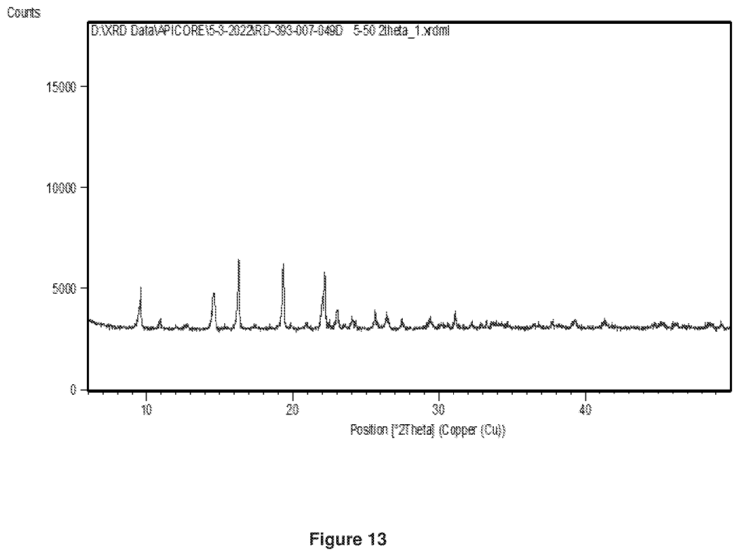

In a seventh embodiment, present invention provides a novel polymorphic form RK5 (Carbon tetrachloride Solvate) of Ferric maltol characterized by XRPD illustrated in FIG. 13.

In a preferred embodiment of the invention, polymorphic form RK5 which is a Carbon tetrachloride Solvate of Ferric maltol can be characterized by XRPD comprising characteristic peaks at 9.6, 14.5, 16.3, 19.3, 22.0 and 22.2±0.2° 2θ.

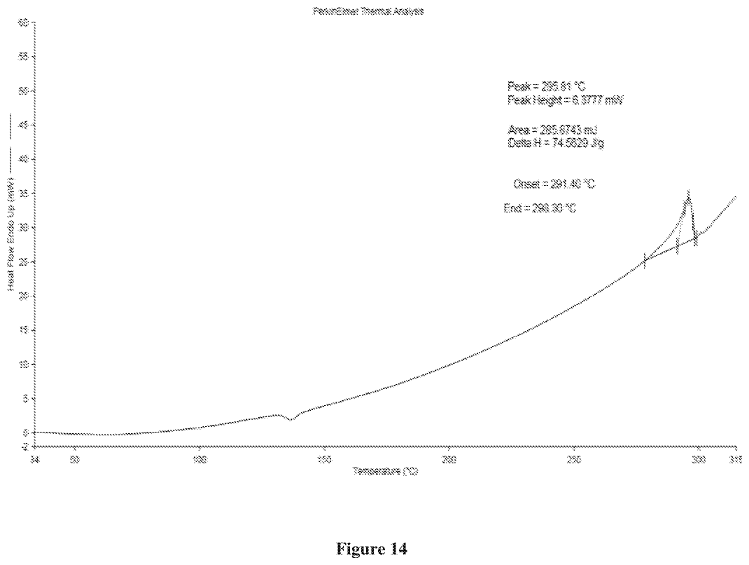

In another embodiment of the invention, polymorphic form RK5 can be characterized by DSC wherein single peak is obtained at 295.8° C. An illustrative DSC thermogram of polymorphic form RK5 is displayed in FIG. 14.

In a further embodiment, present invention provides process for the preparation of polymorphic form RK5 of Ferric maltol comprising the steps of.
a) Slurring the Polymorphic Form Alpha or form RK2 of Ferric maltol in Carbon tetrachloride (CCl₄) at room temperature;
b) Adding Methyl tertiary butyl ether (MTBE) into the slurry mass at room temperature to obtain polymorphic form RK5 of Ferric maltol

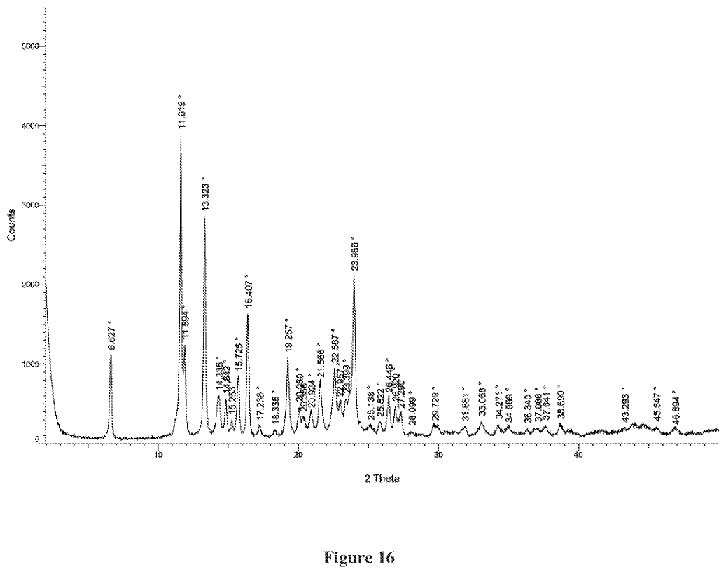

In a eighth embodiment, present invention provides a novel polymorphic form RK6 (Acetic acid hemisolvate) of Ferric maltol characterized by XRPD illustrated in FIG. 16.

In a preferred embodiment of the invention, polymorphic form RK6 which is a Acetic acid hemi solvate of Ferric maltol can be characterized by XRPD comprising characteristic peak at 6.6±0.2° 2θ, and two or more further peaks at 11.6, 13.3, 16.4 and 23.9±0.2° 2θ.

In a further embodiment of the invention, XRPD of polymorphic form RK6 further shows additional peaks at 14.3, 14.8, 16.4, 21.5, 22.5±0.2° 2θ.

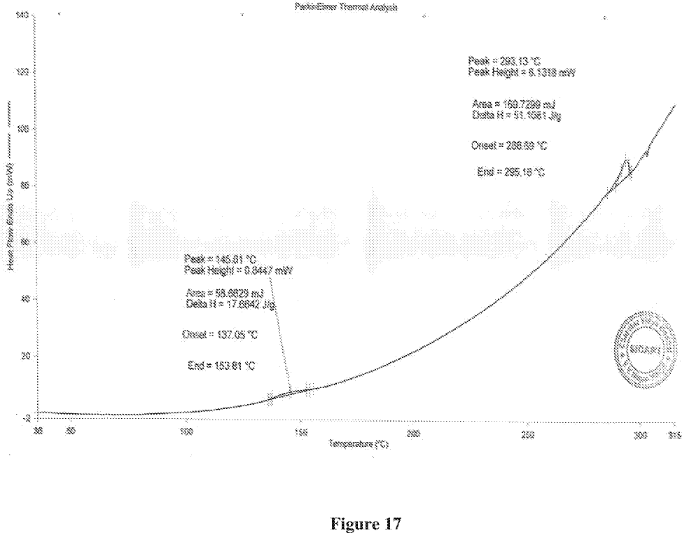

In another embodiment of the invention, polymorphic form RK6 can be characterized by DSC wherein two peaks are obtained at 145.6° C. and 293.1° C. An illustrative DSC thermogram of polymorphic form RK6 is displayed in FIG. 17.

In a further embodiment, present invention provides process for the preparation of polymorphic form RK6 of Ferric maltol comprising the steps of.
a) Slurring dissolving the Polymorphic Form Alpha or RK2 of Ferric maltol in Acetic acid at room temperature;
b) Filtering the solution to remove undissolved particulate;
c) Adding the filtrate slowly into Methyl tertiary butyl ether (MTBE) at room temperature to obtain polymorphic form RK6 of Ferric maltol.

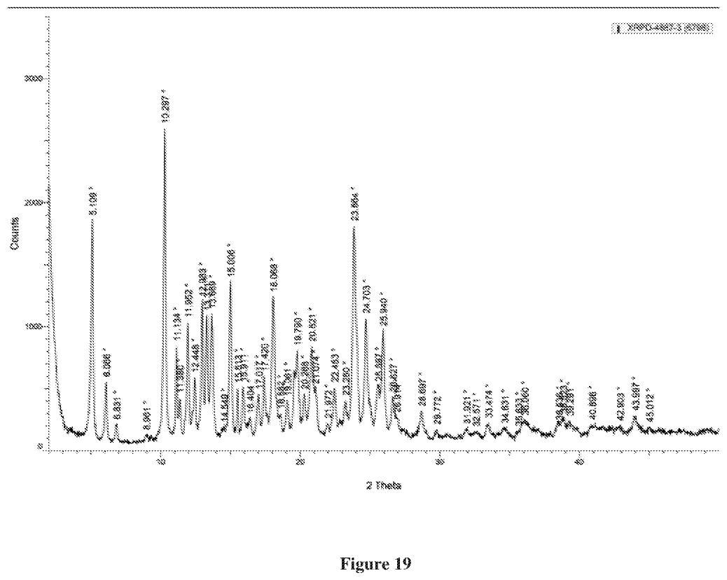

In a ninth embodiment, present invention provides a novel polymorphic form RK7 (Propionic acid hemisolvate) of Ferric maltol characterized by XRPD illustrated in FIG. 19.

In a preferred embodiment of the invention, polymorphic form RK7 which is an Acetic acid hemi solvate of Ferric maltol can be characterized by XRPD comprising characteristic peaks at 5.1, 6.0, 10.3, 13.0, 15.0, 18.0, and 23.8±0.2° 2θ.

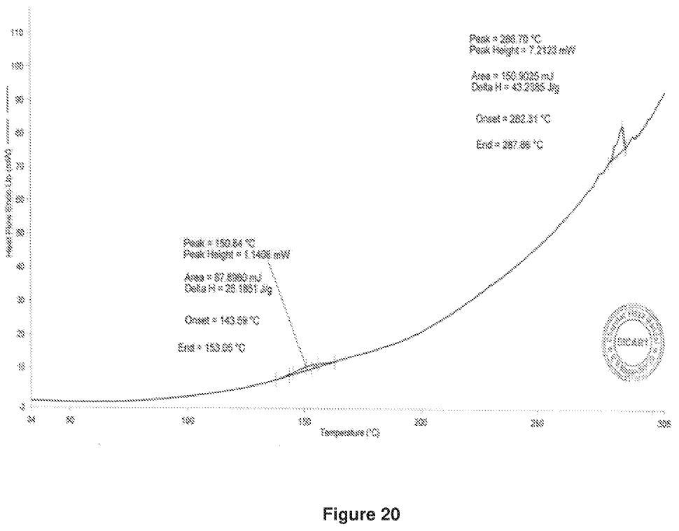

In another embodiment of the invention, polymorphic form RK7 can be characterized by DSC wherein two peaks are obtained at 150.8° C. and 286.7° C. An illustrative DSC thermogram of polymorphic form RK7 is displayed in FIG. 20.

In a further embodiment, present invention provides process for the preparation of polymorphic form RK7 of Ferric maltol comprising the steps of:
a) Slurring/dissolving the Polymorphic Form Alpha or RK2 of Ferric maltol in Propionic acid at room temperature;
b) Filtering the solution to remove undissolved particulate;
c) Adding the filtrate slowly into Methyl tertiary butyl ether (MTBE) at room temperature to obtain polymorphic form RK7 of Ferric maltol.

In another embodiment of the invention, obtained ferric maltol is having purity of greater than or equal to about 95%, 96%, 97%, 98%, 99%, 99.99%,

In a preferred embodiment of the invention, obtained ferric maltol is 99.94% to 99.99% pure.

In a further embodiment of the invention, the yield of the ferric maltol is greater than 50%, preferably 50-90%, more preferably 60-90%, most preferably 80-90% based on the starting materials.

In another embodiment, the present invention also provides the pharmaceutical composition comprising ferric maltol and at least one pharmaceutical excipient.

In a further embodiment, the present invention provides the pharmaceutical composition comprising ferric maltol and at least one pharmaceutical excipient, wherein the dosage form is in the form of tablet or capsule.

Certain specific aspects and embodiments of the present application will be explained in more detail with reference to the following examples, which are provided only for purposes of illustration and should not be construed as limiting the scope of the present application in any manner.

While the foregoing pages provide a detailed description of the preferred embodiments of the invention, it is to be understood that the summary, description and examples are illustrative only of the core of the invention and non-limiting. Furthermore, as many changes can be made to the invention without departing from the scope of the invention, it is intended that all material contained herein may be interpreted as mere illustrative of the invention and not in a limiting sense.

### EXAMPLES

### Example 1: Preparation of Ferric Maltol Technical

Methanol (10V) was charged into a round bottom flask. Sodium hydroxide (47.55 gm; 3 eq.) was then charged into a round bottom flask and stirred at room temperature to dissolve it completely. Maltol (149.8 gm; 3 eq.) was then introduced into a round bottom flask and stirred at room temperature and slurry was obtained. The reaction mixture was then cooled up to 10-20° C. Ferric chloride hexahydrate (107 gm; 1 eq.) or ferric chloride anhydrous (64.2 gm; 1 eq.) was then added lot wised to the round bottom flask and stirred at 10-20° C. for 2-3 hours. Solid was obtained, filtered and washed with cold methanol (2V). The material was suck dried well for 1-2 Hours.

### Example 2: Preparation of Polymorphic Form Alpha of Ferric Maltol

Dichloromethane (7V) was introduced into a round bottom flask. The obtained solid from example 1 was then charged in a round bottom flask and stirred well to dissolve the material. Filter through 0.45-micron filter paper to remove sodium chloride from Dichloromethane layer. Methyl tertiary butyl ether (MTBE) (45V) was then added in Dichloromethane solution to obtain form alpha.
Yield: 60-80% Purity: 99.99%

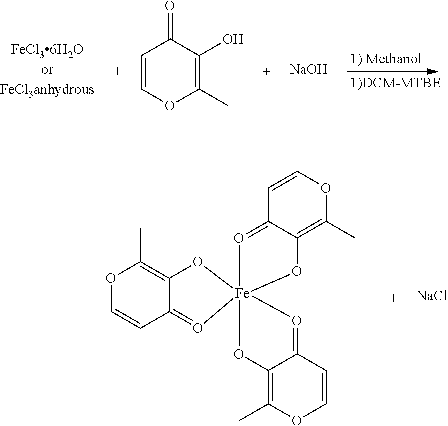

Stress Stability study of Polymorphic Form Alpha was carried out under conditions tabulated in Table 1 given below.

TABLE 1
Stress Stability study of Polymorphic Form Alpha

| Sr. No. | Conditions | PXRD |
|---|---|---|
| 1. | Drying at 30° C.; 3 h | Form Alpha |
| 2. | Drying at 70° C.; 3 h | Form Alpha |
| 3. | Drying at 100° C.; 3 h | Form Alpha |
| 4. | 90% RH; 15 h | Form Alpha |
| 5. | 75% RH; 15 h | Form Alpha |
| 6. | Hexane Slurry | Form Alpha |
| 7. | Grinding; 10 min | Form Alpha |
| 8. | Pressure study (17 Kg/cm2; 15 min) | Form Alpha |

XRPD pattern of Polymorphic Form Alpha of ferric maltol after subjecting it to various stress conditions as mentioned in the Table 1 was found to be stable and consistent with XRPD pattern of Polymorphic Form Alpha of ferric maltol.

### Example 3: Preparation of Polymorphic Form Alpha of Ferric Maltol

Dichloromethane (15 V) was charged into a round bottom flask. Sodium hydroxide (20 gm; 3 eq.) was then introduced into the round bottom flask and stirred at room temperature. Maltol (63 gm; 3 eq.) was then charged into the flask and stirred at room temperature and slurry was obtained. The reaction mass was cooled up to 10-20° C. Ferric chloride hexahydrate (45 gm; 1 eq.) or ferric chloride anhydrous (27 gm; 1 eq.) was then charged lot wise into a flask and stirred at 10-20° C. for 2-3 hours (wine red coloured solution was formed). The Dichloromethane layer was then decanted to separate sodium chloride. The reaction mixture was then filtered through 0.45-micron filter paper to remove traces of sodium chloride or optionally water wash to Dichloromethane layer. Methyl tertiary butyl ether (MTBE) (50V) was then added in Dichloromethane layer and stirred to obtain solid. Material was filtered and dried at 50° C. for 5 hours to obtain polymorphic form alpha of ferric maltol.
Yield: 80-90%. Purity: 99.99%

### Example 4:Preparation of Polymorphic Form RK1 of Ferric Maltol

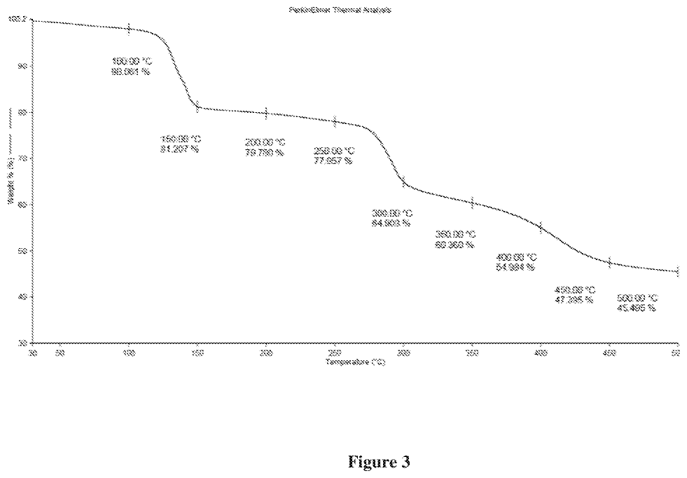

Chloroform (10V) and ethanol (2V) were charged into a round bottom flask. Ferric Maltol (10 gm) was then introduced into a round bottom flask and stirred at room temperature to dissolve it completely. The solution was filtered to remove undissolved particulate. Methyl tertiary butyl ether (MTBE) (45V) was then added in filtered solution at room temperature to obtain form RK1. Obtained solid was filtered and washed with MTBE (2V). Solid was further dried in vacuum oven at 40-50° C. for 5 hours to obtain polymorphic form RK1 of ferric maltol. XRPD, DSC and TGA of polymorphic form RK1 of Ferric Maltol are shown in FIG. 1, FIG. 2 and FIG. 3 respectively. XRPD analysis shows that the solid obtained is consistent with polymorph Form RK1. DSC and TGA results suggest that the solid exists a solvated form.
Yield: 9.8 gm Purity: 99.95%

### Example 5:Preparation of Polymorphic Form RK1 of Ferric Maltol

Chloroform (10V) was charged into a round bottom flask. Ferric Maltol (10 gm) was then introduced into a round bottom flask and stirred at room temperature to dissolve it completely. The solution was filtered to remove undissolved particulate. Ethyl acetate (10V) was then added in filtered solution at room temperature to obtain form RK1. Obtained solid was filtered and washed with ethyl acetate (2V). Solid was further dried in vacuum oven at 40-50° C. for 5 hours to obtain polymorphic form RK1 of ferric maltol
Yield: 9.6 gm Purity: 99.96%

### Example 6: Preparation of Polymorphic Form RK2 of Ferric Maltol

Polymorphic form RK1 of Ferric Maltol obtained in the Example 4 was dried at 100° C. for 3 hours to obtain polymorphic form RK2 of ferric maltol.
Yield: 80% Purity: 99.95%

### Example 7: Preparation of Polymorphic Form RK2 of Ferric Maltol

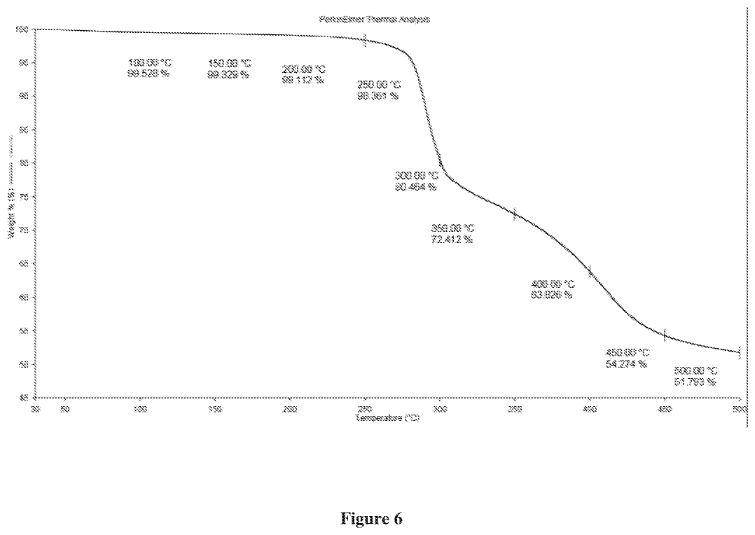

Polymorphic form RK1 of Ferric Maltol obtained in the Example 4 was dried at 150° C. for 3 hours to obtain polymorphic form RK2 of ferric maltol. XRPD, DSC and TGA of polymorphic form RK2 of Ferric Maltol are shown in FIG. 4, FIG. 5 and FIG. 6 respectively. XRPD analysis shows that the solid obtained is consistent with polymorph Form RK2.
Yield: 80% Purity: 99.95%

Stress Stability study of Polymorphic Form RK2 was carried out under conditions tabulated in Table 2 given below.

TABLE 2
Stress Stability study of Polymorphic Form RK2

| Sr. No. | Conditions | XRPD |
|---|---|---|
| 1. | Drying at 150° C.; 3 h | Form RK2 |
| 2. | Drying at 100° C.; 3 h | Form RK2 |
| 3. | 90% RH; 12 h | Form RK2 |
| 4. | 75% RH; 12 h | Form RK2 |
| 5. | 50% RH; 12 h | Form RK2 |
| 6. | MTBE Slurry; 12 h | Form RK2 |
| 7. | Micronization study | Form RK2 |
| 8. | Pressure study (Pressure: 17.5 Kg/cm²) | Form RK2 |

XRPD pattern of Polymorphic Form RK2 of ferric maltol after subjecting it to various stress conditions as mentioned in the Table 2 was found to be stable and consistent with XRPD pattern of Polymorphic Form RK2 of ferric maltol.

### Example 8: Preparation of Polymorphic Form RK3 (Dichloromethane Solvate) of Ferric Maltol

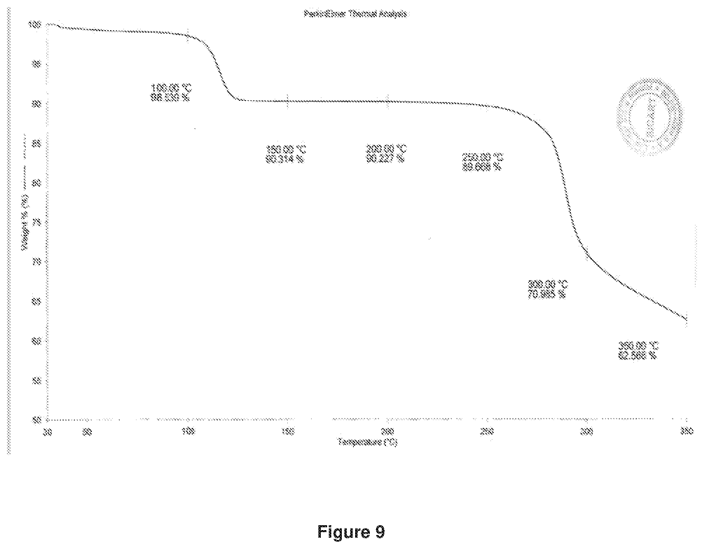

Dichloromethane (3V) were charged into a round bottom flask. Ferric Maltol (10 gm) was then introduced into a round bottom flask and stirred at room temperature. Dark coloured slurry was observed. After 5 min, the solid got precipitated out as Polymorphic form RK3. Methyl tertiary butyl ether (MTBE) (20V) was then added in slurry mass at room temperature. Obtained solid was filtered and washed with MTBE (2V). Solid was further dried in vacuum oven at 40-50° C. for 4-5 hours to obtain polymorphic form RK3 of ferric maltol. XRPD, DSC and TGA of polymorphic form RK3 of Ferric Maltol are shown in FIG. 7, FIG. 8 and FIG. 9 respectively. DSC and TGA results confirms that the solid exists as a solvated form.
Yield: 9.9 gm Purity: 99.95%

### Example 9: Preparation of Polymorphic Form RK4 (Chlorobenzene Solvate) of Ferric Maltol

Ferric maltol (5 gm) was dissolved in dichloromethane (10V) at room temperature into a round bottom flask. The solution was filtered to remove undissolved particulate. The filtrate solvent was then evaporated on rotary evaporator to obtain solid as polymorphic form alpha of Ferric Maltol. Chlorobenzene (10V) was then charged into the flask having polymorphic form alpha and stirred at room temperature. After 5 min, the solid got precipitated out. MTBE (10V) was then added in slurry mass at room temperature. Obtained solid was filtered and washed with MTBE (2V). Solid was further dried in vacuum oven at 40-50° C. for 4-5 hours to obtain polymorphic form RK4 of ferric maltol
Yield: 4.2 gm

### Example 10: Preparation of Polymorphic Form RK4 (Chlorobenzene Solvate) of Ferric Maltol

Ferric maltol (5 gm) was dissolved in chloroform (10V) at room temperature into a round bottom flask. The solution was filtered to remove undissolved particulate. The filtrate solvent was evaporated on rotary evaporator to obtain solid as polymorphic form alpha of Ferric Maltol. Chlorobenzene (10V) was charged into flask having polymorphic form alpha and stirred at room temperature. After 5 min, the solid got precipitated out as polymorphic form RK4. MTBE (10V) was then added in slurry mass at room temperature. Obtained solid was filtered and washed with MTBE (2V). Solid was further dried in vacuum oven at 40-50° C. for 4-5 hours to obtain polymorphic form RK4 of ferric maltol
Yield: 4 gm

### Example 11: Preparation of Polymorphic Form RK5 (CCl₄ Solvate) of Ferric Maltol

Polymorphic form alpha/RK2 (5 gm) of Ferric maltol was slurred in carbon tetrachloride (5V) at room temperature into a round bottom flask. After 30 min, the solid got precipitated out as polymorphic form RK5. MTBE (10V) was then added in slurry mass at room temperature. Obtained solid was filtered and washed with MTBE (2V). Solid was further dried in vacuum oven at 40-50° C. for 4-5 hours to obtain polymorphic form RK5 of ferric maltol.

### Example 12: Preparation of Polymorphic Form RK6 (Acetic Acid Hemi Solvate) of Ferric Maltol

Polymorphic form alpha/RK2 (5 gm) of Ferric maltol was dissolved in acetic acid (5V) at room temperature into a round bottom flask. Reaction mixture was stirred to dissolve the material completely. The solution was then filtered to remove undissolved particulate. The filtrate was added slowly into Methyl tertiary butyl ether (MTBE) (30V) at room temperature to obtain polymorphic form RK6. Obtained solid was filtered and washed with MTBE (2V). Solid was further dried in vacuum oven at 40-50° C. for 4-5 hours to obtain polymorphic form RK6 of ferric maltol.
Yield: 70-90%

### Example 13: Preparation of Polymorphic Form RK6 (Acetic Acid Hemi Solvate) of Ferric Maltol

Polymorphic form alpha/form RK2 (140 gm) of ferric maltol was dissolved in acetic acid (4V) at room temperature into a round bottom flask. Reaction mixture was stirred to dissolve the material completely. The solution was then filtered to remove undissolved particulate. The filtrate was slowly added into Methyl tertiary butyl ether (MTBE) (20V) at room temperature to obtain polymorphic form RK6. Obtained solid was filtered and washed with MTBE (2V). Solid was further dried in vacuum oven at 40-50° C. for 4-5 hours to obtain polymorphic form RK6 of ferric maltol.
Yield: 70-90%
   Stress stability study of polymorphic form RK6 was carried out under various conditions mentioned in Table 3.

TABLE 3
Stress Stability study of polymorphic form RK6

| Sr. No. | Conditions | PXRD |
|---|---|---|
| 1. | Drying at 30° C.; 3 h | Form RK6 |
| 2. | Drying at 70° C.; 3 h | Form RK6 |
| 3. | Drying at 100° C.; 3 h | Form RK6 |
| 4. | 90% RH; 15 h | Form RK6 |
| 5. | 75% RH; 15 h | Form RK6 |
| 6. | Hexane Slurry | Form RK6 |
| 7. | Grinding; 10 min | Form RK6 |
| 8. | Pressure study (17 Kg/cm2; 15 min) | Form RK6 |

XRPD pattern of Polymorphic Form RK6 of ferric maltol after subjecting it to various stress conditions as mentioned in the Table 3 was found to be stable and consistent with XRPD pattern of Polymorphic Form RK6 of ferric maltol.

### Example 14: Preparation of Polymorphic Form RK7 (Propionic Acid Hemi Solvate) of Ferric Maltol

Polymorphic form alpha/form RK2 (5 gm) of Ferric maltol was dissolved in propionic acid (10V) at 45° C. into a round bottom flask. Reaction mixture was stirred to dissolve the material completely. The solution was then filtered to remove undissolved particulate and cooled to room temperature. The filtrate was slowly added into Methyl tertiary butyl ether (MTBE) (40V) at room temperature to obtain polymorphic form RK7. Obtained solid was filtered and washed with MTBE (2V). Solid was further dried in vacuum oven at 40-50° C. for 4-5 hours to obtain polymorphic form RK7 of ferric maltol.
Yield: 80-90%

Without wishing to be bound to a theory, the process described in the present invention is believed to be an improved process for the preparation of ferric maltol which is commercially scalable, economical, stable and provides novel crystalline forms of ferric maltol with a characteristic XRPD pattern, and in some cases with a single crystal form.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1-6. (canceled)

7. A Polymorphic form RK1 of Ferric maltol characterized by X-ray powder diffraction pattern comprising a characteristic crystalline peaks expressed in degrees 2-theta at 5.4, 11.5, 12.5, 16.0, 16.4, 23.4 and 24.2±0.2°

8. An improved process for the preparation of polymorphic form RK1 of ferric maltol as claimed in claim 7, comprising the steps of:
a) providing a solution of ferric maltol in chloroform or mixture of chloroform and organic solvent;
b) addition of anti-solvent to the solution obtained in step a);
c) isolation of polymorphic form RK1 of Ferric maltol.

9. An improved process as claimed in claim 8, wherein organic solvent used in step a) is ethanol and anti-solvent used in step b) is MTBE or ethyl acetate.

10. A Polymorphic Form RK2 of Ferric maltol characterized by X-ray powder diffraction pattern comprising characteristic crystalline peaks expressed in degrees 2-theta at 6.8, 10.6, 12.2, 13.8, 15.2, 15.8, 22.9, 24.2±0.2°

11. An improved process for the preparation of polymorphic form RK2 of ferric maltol as claimed in claim 10, comprising:
a) drying the polymorphic form RK2 of ferric maltol between 100° C.-150° C. for 3-4 hours.

12. A polymorphic form RK3 of Ferric maltol characterized by X-ray powder diffraction pattern comprising characteristic crystalline peaks expressed in degrees 2-theta at 11.5, 14.2, 18.3, 23.3, 24.9±0.2°.

13. An improved process for the preparation of polymorphic form RK3 of Ferric maltol as claimed in claim 12, comprising the steps of:
a) Slurring the Ferric maltol in Dichloromethane at room temperature;
b) Adding Methyl tertiary butyl ether into the slurry mass at room temperature to obtain polymorphic form RK3 of Ferric maltol

14. A polymorphic form RK4 of Ferric maltol characterized by X-ray powder diffraction pattern comprising characteristic crystalline peaks expressed in degrees 2-theta at 9.1, 11.9, 13.6, 17.1, 22.0 and 22.7±0.2°.

15. An improved process for the preparation of polymorphic form RK4 of Ferric maltol as claimed in claim 14, comprising the steps of:
a) Dissolving Ferric maltol in Dichloromethane or Chloroform at room temperature;
b) Filtering the solution to remove undissolved particulate;
c) evaporating the solvent from the filtrate on rotary evaporator to obtain Polymorphic form Alpha of ferric maltol;
d) Adding Chlorobenzene and stirring at room temperature for 5-10 minutes;
e) Adding Methyl tertiary butyl ether (MTBE) into the slurry mass at room temperature to obtain polymorphic form RK4 of Ferric maltol.

16. A polymorphic form RK5 of Ferric maltol characterized by X-ray powder diffraction pattern comprising characteristic crystalline peaks expressed in degrees 2-theta at 9.6, 14.5, 16.3, 19.3, 22.0 and 22.2±0.2°.

17. An improved process for the preparation of polymorphic form RK5 of Ferric maltol as claimed in claim 16, comprising the steps of:
a) Slurring Polymorphic Form Alpha or RK2 of Ferric maltol in Carbon tetrachloride (CCl₄) at room temperature.
b) Adding Methyl tertiary butyl ether (MTBE) into the slurry mass at room temperature to obtain polymorphic form RK5 of Ferric maltol.

18. A polymorphic Form RK6 of Ferric maltol characterized by powder X-ray diffraction pattern comprising a characteristic crystalline peak expressed in degrees 2-theta at 6.6±0.2°, and two or more further peaks expressed in degrees 2-theta selected from 11.6, 13.3, 16.4 and 23.9±0.2° 2θ.

19. (canceled)

20. An improved process for the preparation of polymorphic form RK6 of Ferric maltol as claimed in claim 18 comprising the steps of:
a) Dissolving the Polymorphic Form Alpha or RK2 of Ferric maltol in Acetic acid at room temperature;
b) Filtering the solution to remove undissolved particulate;
c) Adding the filtrate slowly into Methyl tertiary butyl ether (MTBE) at room temperature to obtain polymorphic form RK6 of Ferric maltol.

21. A polymorphic form RK7 of Ferric maltol characterized by X-ray powder diffraction pattern comprising characteristic crystalline peaks expressed in degrees 2-theta at 5.1, 10.3, 13.0, 15.0, 18.0, and 23.8±0.2°.

22. An improved process for the preparation of polymorphic form RK7 of Ferric maltol as claimed in claim 21, comprising the steps of:
a) Slurring/dissolving the Polymorphic Form Alpha or RK2 of Ferric maltol in Propionic acid at room temperature;
b) Filtering the solution to remove undissolved particulate;
c) Adding the filtrate slowly into Methyl tertiary butyl ether (MTBE) at room temperature to obtain polymorphic form RK7 of Ferric maltol.
